(19) United States
(12) Patent Application Publication
(10) Pub. No.: US 2021/0110916 A1
(43) Pub. Date: Apr. 15, 2021

(54) SYSTEMS AND METHODS FOR MATCHING A PROVIDER TO A USER

(71) Applicant: Andrino D. Flevotomos, Clifton, VA (US)

(72) Inventor: Andrino D. Flevotomos, Clifton, VA (US)

(21) Appl. No.: 16/599,583

(22) Filed: Oct. 11, 2019

(51) Int. Cl. G16H 40/20; G16H 10/60; G06F 16/2457

(52) U.S. Cl. CPC G16H 40/20; G06F 16/24578; G16H 10/60

(57) ABSTRACT

The present disclosure generally relates to healthcare resources and, more particularly, to systems and methods for matching a provider to a user. A method for matching providers includes: obtaining standardized user data from a user; obtaining standardized provider data from providers; generating a list of matching providers by comparing the standardized user data to the standardized provider data; and narrowing the list of the matching providers by obtaining additional data from the user and comparing the additional data to the standardized provider data.

### TECHNICAL FIELD

The present disclosure generally relates to healthcare resources and, more particularly, to systems and methods for matching a provider to a user.

### BACKGROUND

Finding an appropriate healthcare provider can be problematic. While an individual may able to read online reviews and search provider websites, there can be uncertainty on whether the information is accurate and/or unbiased. Accordingly, the patient might not be able to determine the appropriate healthcare provider for their needs based on this limited information. In other situations, a patient may not even know the type of healthcare services that they should seek, let alone healthcare providers which provide these services.

### SUMMARY OF INVENTION

In accordance with a first aspect of the invention, a method for matching providers comprises: obtaining standardized user data from a user; obtaining standardized provider data from providers; generating a list of matching providers by comparing the standardized user data to the standardized provider data; and narrowing the list of the matching providers by obtaining additional data from the user and comparing the additional data to the standardized provider data.

In accordance with another aspect of the invention, a computer system for matching healthcare providers comprises: a hardware memory device that stores program instructions of a matching tool; and a hardware processor that executes the program instructions and causes the matching tool to: obtain user data from a user; standardize the user data into a format; assign values to the standardized user data in order to prioritize the standardized user data; obtain provider data from providers; standardize the provider data into the format; generate a list of matching providers by comparing the prioritized user data to the provider data; and narrow the list of the matching providers by obtaining additional data from the user and prioritizing the additional data.

In accordance with another aspect of the invention, a computer program product for matching healthcare providers, the computer program product comprising computer readable program instructions stored on computer readable storage medium, the computer readable program instructions being executable on a computing device to cause the computing device to: obtain user data from a user; weight the user data in order of importance to the user; obtain provider data from providers; generate a list of matching providers by comparing the weighted user data to the provider data; and calibrate the list of the matching providers by obtaining additional data from the user and assigning weights to the additional data.

### BRIEF DESCRIPTION OF THE SEVERAL VIEWS OF THE DRAWINGS

The present invention is described in the detailed description which follows, in reference to the noted plurality of drawings by way of non-limiting examples of exemplary embodiments of the present invention.

FIG. 1 is an illustrative architecture of a computing system in accordance with aspects of the invention;

FIG. 2 shows an exemplary framework for matching a healthcare provider in accordance with aspects of the invention; and

FIG. 3 shows an exemplary process for matching a healthcare provider in accordance with aspects of the invention.

### DETAILED DESCRIPTION OF ASPECTS OF THE INVENTION

The present disclosure generally relates to healthcare resources and, more particularly, to systems and methods for matching a provider to a user. In embodiments, the systems and methods described herein can assist users in obtaining recommendations for healthcare services and corresponding healthcare providers. Advantageously, the user can receive an appropriate provider to address their healthcare needs.

In searching for a healthcare provider, a person may perform a search based on location and/or availability of the provider. However, this type of search does not account for things which may be desired by the person. As an example, a person may want a healthcare provider which matches their expectations for empathy, compassion, risk adversity, amenability to holistic approaches and tolerance for anxious clients/patients, amongst other examples. Additionally, a person may be unaware of healthcare services which are available or which they should consider in order to fulfil their healthcare goals.

Online information of healthcare providers, such as reviews and provider websites, generally do not address the above. Specifically, the information listed on the provider website may be minimal, and may not provide information regarding the healthcare provider's approaches in practicing healthcare. Additionally, an online review of a provider is essentially an opinion of an anonymous individual. Accordingly, the online review may be unreliable. Alternatively, people generally have less time to interview multiple providers in order to determine who might serve them best. In this way, a person may not find the appropriate healthcare provider for them. However, research has shown that the synergy between provider and patient is a determinant in the overall success of healthcare. Accordingly, the systems and processes described herein are intended to address these circumstances.

The systems and processes of the present disclosure provide the practical application of matching a user to a healthcare provider based on a calibrated process which accounts for various attributes desired by the user. In embodiments, the systems and processes described herein are implemented in various phases for both the user and the provider in order to provide a precise calibrated matching between the user and healthcare provider. In embodiments, phase 1 includes data collection for both the user and the healthcare provider to initiate the matching processes. Phase 2 for the user includes providing data for additional/ancillary services needed, while phase 2 for the provider includes providing data for detailed and personal demographical questions. Phase 3 for the user includes executing a personality test, while phase 3 for the provider includes providing data for scenario based behavioral questions. Phase 4 is directed to the provider and includes the provider executing a personality test.

Accordingly, the structures and processes described herein are directed to a network-based patient and provider matching system and method that collects, converts and consolidates patient information from various users into a standardized format. Further, the matching system and method collects, converts and consolidates provider information from various health-care providers into a standardized format. The matching system and method stores the standardized patient information and standardized provider information in network-based storage devices, and generates matches between the user and the health care providers based on the standardized information from the users and the providers.

The systems and processes described herein leverage the above data in order to provide a mechanism for obtaining accurate calibrated matches for healthcare services. Further, the data that is provided is not only provided by a user seeking healthcare, but also provided by the healthcare provider themselves. In this way, the systems and processes of the present disclosure provide accurate matching between the user and the healthcare provider based on information relevant to both the patient and the provider while ignoring online ratings of the providers which may be biased.

Although the systems and methods described hereafter are with regard to exemplary processes, and/or computer program products, it should be understood that other implementations are also contemplated by the present disclosure as described herein. For example, other devices, systems, appliances, and/or computer program products according to embodiments of the present disclosure will be or become apparent to one of ordinary skill in the art upon review of the drawings and detailed description. It is intended that all such additional other devices, systems, appliances, processes, and/or computer program products be included within the scope of the present disclosure.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects. Furthermore, aspects of the present disclosure may take the form of a computer program product embodied in one or more computer readable storage medium(s) having computer readable program code embodied thereon.

The computer readable storage medium (or media) having computer readable program instructions thereon causes one or more computing processors to carry out aspects of the present disclosure. The computer readable storage medium can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

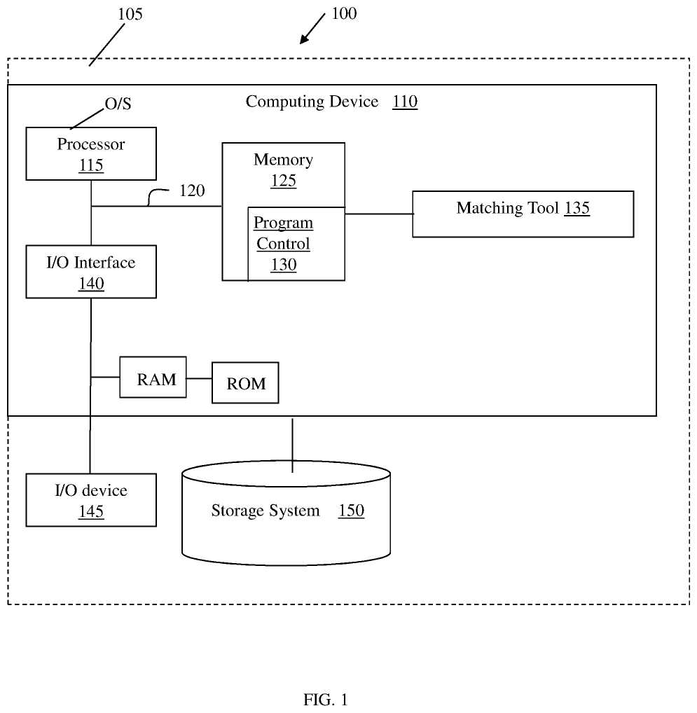

A non-exhaustive list of more specific examples of the computer readable storage medium includes the following non-transitory signals and mediums: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. The computer readable storage medium is not to be construed as transitory signals per se; instead, the computer readable storage medium is a physical medium or device which stores the data. The computer readable program instructions may also be loaded onto a computer, for execution of the instructions, as shown in FIG. 1.

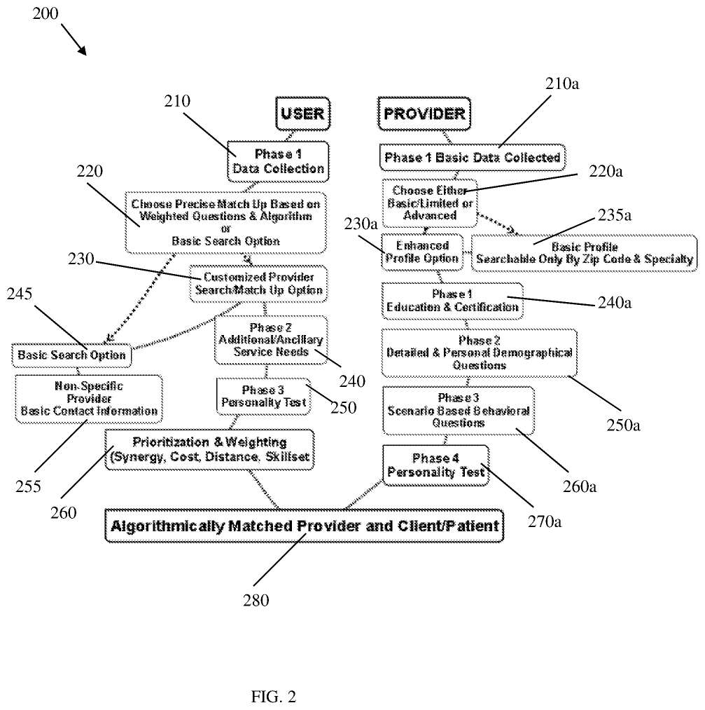

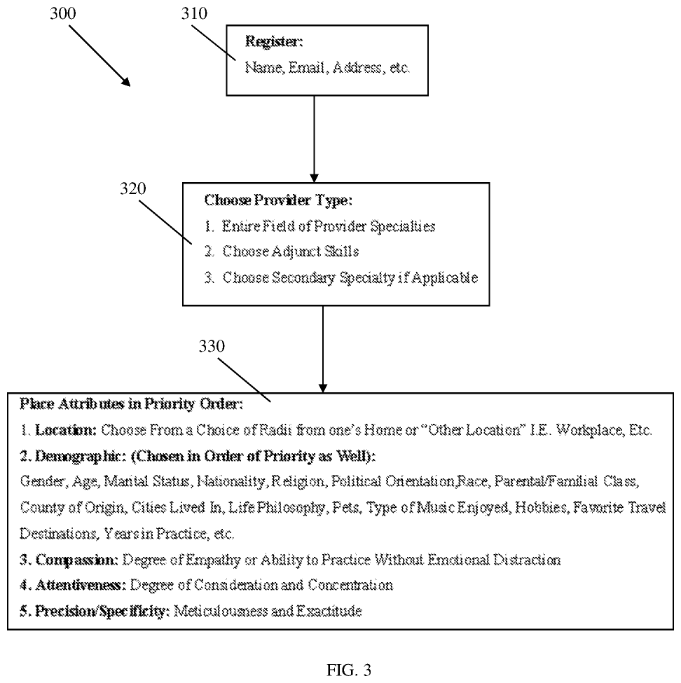

FIG. 1 shows a computer infrastructure 100 for implementing steps in accordance with aspects of the disclosure. To this extent, the infrastructure 100 can implement the matching between a user and a healthcare provider shown in the matching framework 200 of FIG. 2 and the matching process 300 of FIG. 3. The infrastructure 100 includes a server 105 or other computing system that can perform the processes described herein. In particular, the server 105 includes a computing device 110. The computing device 110 can be resident on a network infrastructure or computing device of a third party service provider (any of which is generally represented in FIG. 1).

The computing device 110 includes a processor 115 (e.g., CPU), memory 125, an I/O interface 140, and a bus 120. The memory 125 can include local memory employed during actual execution of program code, bulk storage, and cache memories which provide temporary storage of at least some program code in order to reduce the number of times code which are retrieved from bulk storage during execution. In addition, the computing device includes random access memory (RAM), a read-only memory (ROM), and an operating system (O/S).

The computing device 110 is in communication with external I/O device/resource 145 and storage system 150. For example, I/O device 145 can comprise any device that enables an individual to interact with computing device 110 (e.g., user interface) or any device that enables computing device 110 to communicate with one or more other computing devices using any type of communications link. The external I/O device/resource 145 may be for example, a handheld device, PDA, handset, keyboard etc.

In general, processor 115 executes computer program code (e.g., program control 130), which can be stored in memory 125 and/or storage system 150. Further, databases containing the data of the user and/or provider, can be stored in the storage system 150. Moreover, in accordance with aspects of the invention, program control 130 controls a matching tool 135, which performs the matching of the user to a healthcare provider described herein. The matching tool 135 can be implemented as one or more program codes in program control 130 stored in memory 125 as separate or combined modules.

Additionally, the matching tool 135 may be implemented as separate dedicated processors or a single or several processors to provide the function of this tool. While executing the computer program code, the processor 115 can read and/or write data to/from memory 125, storage system 150, and/or I/O interface 140. The program code executes the processes of the invention. The bus 120 provides a communications link between each of the components in computing device 110.

The matching tool 135 is utilized to perform the matching framework 200 shown in FIG. 2 and the matching process 300 of FIG. 3. For example, the different data entries, checks, analysis, comparisons and evaluations shown in FIG. 2 can be used to evaluate and match a potential patient to a healthcare provider. In embodiments, the matching tool 135 checks, analyzes, compares, evaluates and assesses a healthcare provider in view of the user's data and the healthcare provider's data.

As an example, a user answers detailed questions regarding a type of provider, sub-specialty of the provider, years of experience of the provider, certification requirements of the provider, additional services offered by the provider. Additionally, the user will complete a comprehensive personality questionnaire to further determine the attributes of the provider desired by the user. The above user data can be entered through a graphical user interface (GUI) on the computing device 110, with the interface being hardware or a combination of both hardware and software. The user is given remote access through the GUI to view or update the user data using the user's own local device, e.g., computing device 110, in real time.

The above user data is converted into a standardized format to allow for weights to be assigned to the user data. When a user wants to update the records of data, the user can input the update in any format used by the user's local device. Whenever the user information is updated, it will first be converted into a standardized format and then stored in the collection of records on one or more of the network-based storage devices. After the updated user information has been stored, the information will be weighted again.

The above data will be used to match the user with the perfect provider for them in view of the data. In embodiments, providers can also contribute to the data used for matching. For example, the provider can provide data concerning their specialty, sub-specialty, additional services and qualifications/licenses/education/years of experience. In further embodiments, the provider can complete a detailed provider profile questionnaire. Similar to the user data, the provider data will be standardized so that weights can be assigned to the provider data.

Once the data has been collected from both the user and the provider, a matching process 300 shown in FIG. 3 is implemented so that calibrated match between user and provider can be generated. In this way, an ideal provider can be recommended to the user. In further embodiments, another component of the matching tool 135 can also recommend to the user healthcare services which match the user's data. In this way, the systems and processes described herein provide the practical application of using dynamic information for both a user and a healthcare provider to create an ideal calibrated match for the user and provider. In further embodiments, the matching framework 200 of FIG. 2 and the matching process 300 of FIG. 3 are implemented by the matching tool 135 to provide data concerning additional services possibly not considered by the user and/or unknown to the user.

FIG. 2 shows an exemplary matching framework 200 for implementing a calibrated match between a user and a healthcare provider. A user can be defined as an individual or entity who desires healthcare services. A healthcare provider can be an individual or entity who provides healthcare services. Examples of providers include naturopaths, holistic pediatric physicians, holistic primary care physicians (PCPs), homeopaths, chiropractors, physical therapists, cranial sacral practitioners, alternative health care providers, biological dentists, personal trainers, mental health practitioners, certified hypnotists, nutritionists, applied kinesiologists, acupuncturists, massage therapists, iridologists, osteopaths, aromatherapists, reiki practitioners, energy workers, medical intuitives, integrative medicine practices, compounding pharmacists and functional medicine specialists, amongst other examples.

Matching framework 200 begins with Phase 1 at a first data collection step 210 for the user and at step 210a for the healthcare provider. At step 210, the user answers questions regarding their personal information and healthcare needs, including user name, email address, home and/or work address, a type of provider desired, a specialty and/or sub-specialty of the provider, years of experience of the provider and preferred certification requirements acquired by the provider, amongst other examples. At step 210a, providers can also answer questions directed to the provider's healthcare services, including their specialty and sub-specialty, additional services offered and qualifications/licenses/education/years of experience, amongst other examples.

At step 220, the user selects which matching option they desire to implement. Specifically, the user will choose whether to implement a precise match up based on weighted questions and process 300, or a basic search option. If the user selects to implement the customized provider search/match up (precise calibrated matching) option at step 230, the user will continue along onto phase 2 and phase 3 of steps 240, 250, 260, 270 and 280, in order to be precisely matched with providers based on the calibrated process 300.

Alternatively, if the user selects the basic search option at step 245, the user will be provided with a basic provider match at step 255 and the processes of matching framework 200 will end. In embodiments, the provider match at step 255 can be based on the information provided at step 210, i.e., information concerning type of provider, specialty and/or sub-specialty of the provider, years of experience of the provider and preferred certification requirements acquired by the provider, for example.

At step 220a, the provider will select a profile option they would like displayed to potential patients (users). In embodiments, the provider can select either a basic/limited profile or an advanced profile. If the provider selects the advanced profile option at step 230a, the matching framework 200 will continue onto phase 1 of data collection process and also onto phases 2-4 of steps 250a, 260a, 270a and 280, in order to be precisely matched with a potential patient (user) through the matching process 300. Alternatively, if the provider selects the basic profile option at 235a, the processes of the matching framework 200 will end and the provider will be matched only by zip code and specialty.

Continuing with FIG. 2, at step 240 the user implements phase 2 of the matching framework 200. In embodiments, the user will provide data regarding any additional/ancillary services desired by the user. At step 240a, the provider will provide their educational and certification information. At step 250, the user will complete phase 3 of the matching framework 200. In embodiments, the user completes a personality test at step 250 in order to glean their degree of empathy, compassion and risk adversity, amongst other examples. In embodiments, a user's attributes for a healthcare provider can include a degree of empathy desired, an ability for the provider to practice without emotional distraction, a degree of consideration and concentration from the provider and a meticulousness and exactitude from the provider. Further examples of questions for the user personality test include provider gender, provider age, provider marital status, provider nationality, provider religion, provider political orientation, cities the provider has lived in, provider life philosophy, pets owned by the provider, type of music enjoyed by the provider, hobbies of the provider, favorite travel destinations of the provider and years the provider has been in practice, amongst other examples.

Step 250 allows for a user's intangible attributes of a healthcare provider to be utilized in matching the user to the provider. At step 250a, the healthcare provider will answer detailed and personal demographical questions. Examples of personal demographical questions include gender, age, marital status, nationality, religion, political orientation, cities lived in, life philosophy, pets, type of music enjoyed, hobbies, favorite travel destinations and years in practice, amongst other examples.

At step 260, the user's answers from the previous step will be standardized so that the user data can be weighted. The standardized user data is then analyzed for prioritization and weighting by the matching process shown of FIG. 3. In this way, the structures and processes described herein include a comprehensive psycho-social matching criteria not driven by cost in any way. In embodiments, the answers of the personality test of step 250 will be weighted based on importance and relevance to the user. For example, if a user provided in the personality test of step 250 that empathy of a healthcare provider is the most important attribute for a provider to have, the matching process will weigh the empathy attribute higher than all other factors, e.g., synergy, cost, distance and skillset, amongst other examples. Accordingly, the remaining attributes will be ranked in descending or ascending order of importance. In this way, the matching of matching framework 200 and matching process 300 occurs through user created variables which are weighted. Specifically, the matching of matching framework 200 and matching process 300 is a comprehensive psycho-social matching which is not driven by any one variable. Instead, the matching is based on weighted variables in view of user determined/chosen factors.

At step 260a, the provider will answer scenario based behavioral questions in order for the matching process to ensure an appropriate match of a healthcare provider to the user. In embodiments, the scenario based behavioral questions can include questions which will glean a healthcare provider's degree of empathy, compassion, risk adversity, amenability to holistic approaches and tolerance for anxious clients/patients, amongst other examples.

As illustrative examples, the questions within the scenario based behavioral questionnaire include:

(1) On the way to the hospital to take care of a serious case where time is of the essence and whom only you can help you witness an accident. Do you: a) Stop and help the critical accident victim? or b) Continue on to the patient desperately awaiting for you?

(2) In dealing with patients/clients, is your inclination to: a) Guide your patient directly towards wellness? or b) Discuss the often meandering emotional details regarding your patient/client's situation?

(3) Do new interactions: a) Stimulate and energize you? or b) Tax your reserves?

(4) Do you feel: a) More consistently high-performing than ingenious? or b) More ingenious than high-performing?

You have awakened with an oral cold sore. Do you prefer: a) Speaking to people whose backs happen to be turned towards you? or b) Speaking directly to their face(s)?

Which is most important to you: a) Your family? b) Your clients/patients? c) Your community reputation you built over many years of struggle and hardship? or d) Your own mental/physical health and welfare?

Would you rather: a) Change to a much lower paying and less glamorous job where you can still help people in less meaningful ways than before? b) Change careers entirely promising gainful employment, but helping only yourself and your family? c) Stop working entirely out of principle? or d) Move to a foreign and uncomfortable country where you can continue your original occupation for free room and board and a minuscule stipend?

Would you say you are more: a) Able to find your way out of precarious situations? b) Able to avoid getting into precarious situation c) Adept at handling the repercussions to mistakes you've made; or d) Averse to the guilt of having made a small error?

What is Most Important to You: a) God? b) Family? c) Survival? or d) Experiencing love?

Someone you know and love has embezzled from you. They did so to help an individual who has manipulated them and surreptitiously forced them to do so. You know and have had a previous falling out with the person that engineered the misdeed. Who is most at fault: a) The person/friend who committed the offense? b) The manipulator with whom you already have a negative history with? c) You? d) Everyone involved? e) Just the perpetrator and the engineer of the wrongdoing?

A scenario based behavioral question for a specific healthcare provider, e.g., midwife, is as follows: Would you rather: 1. Knit? 2. Pet a lap animal? 3. Hold a newborn? or 4. Spend time with your family?

Each question of the above scenario based behavioral questions demonstrate a different character detail. In the above midwife example, while holding a baby may seem like the choice that would make the midwife most universally acceptable to the majority of users performing a search, this may not be the desired character trait of the user using the matching framework 200.

The above records of data are entered through a graphical user interface (GUI) on the computing device 110 for the user and a computing device of the provider. The GUI can be hardware or a combination of both hardware and software. The user is given remote access through the GUI to view or update the user information using the user's own local device (e.g., a personal computer or wireless handheld device). When a user wants to update the records, the user can input the update in any format used by the user's local device. Further, the provider is also given remote access through the GUI to view or update the above provider information using the provider's own local device (e.g., a personal computer or wireless handheld device). When the provider wants to update the records, the provider can input the update in any format used by the provider's local device.

Step 270a is an additional phase 4 that the provider performs by completing a personality test to glean the provider's degree of empathy, compassion, risk adversity, amenability to holistic approaches and tolerance for anxious clients/patients, amongst other examples. In embodiments, the answers to the personality test of step 270a are used in conjunction with the answers of the scenario based behavioral questions from step 260a to ensure a precise and calibrated match of a healthcare provider to the user. The personality test can include questions on the provider's empathy, ability to practice without emotional distraction, the provider's consideration and concentration, and also a meticulousness and exactitude from the provider.

The matching framework 200 concludes with an process match between the user and the provider at step 280. In embodiments, the matching framework 200 will display to the user a list of providers which have been precisely matched. Depending on the provider's selected options, the provider's information can be listed by name and specialty or for a fee the provider can have an advanced profile complete with video introduction, improved interface appearance, links to reviews and websites, amongst other examples. The list of providers is displayed by the graphical user interface (GUI) on the computing device 110.

The matching of user to provider at step 280 occurs by standardizing the user data and ultimately assigning a value to every question/choice of the user standardized data throughout the matching framework 200. The values can change based on how the user has prioritized at least five attributes, although other amounts of attributes are contemplated herein. In this way, each prioritization order that is selected by the user results in uniquely assigned values to their respective attribute and sub-category questions. In embodiments, user choices from certain attributes can be further prioritized for greater stratification. As an example, attributes for location and demographics can be further prioritized, compared to remaining attributes for provider compassion, provider attentiveness and precision/specificity of the provider, amongst other contemplated attributes. More specifically, the location and demographics attributes can be further prioritized based on features within the attributes. For example, if distance to work is more important to the user than a distance from their home, the distance to work feature will be prioritized over the distance to home.

In embodiments, if there are five major categories of attributes, there can be about 120 possible prioritization orders. In further embodiments, the questions and weighting differ based on provider specialty. In other words, every specialty has their own set of questions ranging from 50-250 in quantity and the value of the questions are specific to the specialty. In this way, each specialty can have its own weighting for each prioritization. As an example, 120 prioritization orders x˜50 questions for that specific specialty=˜6k discrete values. The healthcare providers with the highest values in view of the user's data will be matched to the user.

As an example of the user using the matching framework 200 to obtain a precisely matched provider, the user is seeking a midwife for healthcare services. The user's desired provider attribute prioritization is as follows: 5 (precision/specificity), 2 (demographic), 3 (compassion), 1 (location), 4 (attentiveness). Further, the user has also answered their desired sub-attributes for categories 1 (location) and 2 (demographic). In this example, the midwife questions (as answered by the midwife healthcare provider at an earlier time) will be assigned values based on the prioritization selected by the user and also as follows: questions that reference category 5 (precision/specificity) will be equal to a range of about 1.8 points (pts) to −1.8 pts depending on how they are answered; questions that reference category 2 (demographic) will be equal to a range of about 1.6 pts to −1.6 pts depending on how they are answered; questions that reference category 3 will be equal to a range of about −1.2 pts to 1.2 pts depending on how they are answered; questions that reference category 1 will be equal to a range of about 1.0 pts or zero pts depending on how they are answered; and questions that reference category 4 will be equal to a range of about 0.50 pts or zero pts depending on how they are answered. In embodiments, not all questions are binary. Further, some answers are equal to or less than the highest point value, but may still have a positive (+) score.

Based on the above results/scoring, an initial list of top choices of providers are derived. At this point, additional calibration questions can be generated and values to their answers can be tabulated to further calibrate the user's responses to ensure accuracy of the answers already provided. These additional calibration questions can be additional circumlocutory questions to determine if the user answered the previous questions with or without self-judgement or internal bias, amongst other examples. In embodiments, these additional calibration questions can be further personality questions, similar to the questions of the personality test at step 250. These additionally calibration questions will then narrow down the initial list of healthcare providers and match the provider with the highest score to the user in order to present the most appropriate provider for the user.

The questions of matching framework 200 are respective to the user's desired criteria, and are not directed to a specific provider. In this way, there is no right or wrong answer by the user. Additionally, any provider attempting to artificially answer questions so as to be more marketable within the systems and processes described herein will be unable to do so, since the answers provided by the user are confidential and unknown to the provider. In this way, the structures and processes described herein provide the practical application of a matching a provider based on user's desired traits and attributes without any outside influence.

Accordingly, the structures and processes described herein recite a combination of additional elements including storing information, providing remote access over a computing network, converting the user and/or provider data and the updated user and/or provider data which is in a non-standardized form into a standardized form, amongst other additional elements. Specifically, the additional elements recite a specific improvement over conventional systems by allowing remote users and providers to share information in real time in a standardized format, regardless of the format in which the information was input by the user and/or provider.

FIG. 3 illustrates a matching process 300 implemented by the matching tool 135 of FIG. 1 and the matching framework 200 of FIG. 2 for matching the user with the healthcare provider. At step 310, the matching process 300 obtains the user registration information from step 210 of FIG. 2. The information obtained at step 310 includes the user name, email address and home and/or work address, amongst other examples. At step 320, the matching process 300 obtains the provider type information from step 210 of FIG. 2. The information obtained at step 320 includes the field of provider specialties desired, adjunct skills of the provider and secondary specialty of the provider, if applicable.

At step 330, the matching process 300 places the answers of the personality test of step 250 of FIG. 2 in a priority order. The answers cover various topics, including provider location, provider demographics, provider compassion, provider attentiveness and provider precision/specificity. Examples of provider location include a radius from a user's home or “other location,” i.e., workplace. Examples of provider demographics include provider gender, provider age, provider marital status, provider nationality, provider religion, provider political orientation, cities the provider has lived in, provider life philosophy, pets owned by the provider, type of music enjoyed by the provider, hobbies of the provider, favorite travel destinations of the provider and years the provider has been in practice, amongst other examples. Examples for provider compassion includes a degree of empathy possessed by the provider or an ability for the provider to practice without emotional distraction, amongst other examples. Examples for provider attentiveness includes a degree of consideration possessed by the provider and concentration from the provider, amongst other examples. Examples for provider precision/specificity include a meticulousness and exactitude from the provider, amongst other examples.

In embodiments, the matching process 300 will use the answers from the matching framework 200 from both the users and the healthcare providers to generate an initial list of providers to the user. The matching process 300 will then provide the user with additional questions to further calibrate their responses. These are circumlocutory questions to determine if the user answered the questions of the matching framework 200 with or without self-judgement or internal bias. With the answers to the additional questions, the matching framework 200 is further calibrated in view of the user's desires and while narrow down the list of providers down to a provider which matches the most with the user's desired attributes.

In this way, the structures and processes described herein allow for the streamlining of documents and/or sources used in selecting a healthcare providing by matching the healthcare provider to the user based on desired attributes by the user. Additionally, this matching is further calibrated to ensure that a precise matching was achieved by requesting additional information from the user and calibrating the match based on this additional information. Accordingly, the structures and processes described herein reduce the amount of steps that a user must complete in selecting a healthcare provider. Further, the structures and processes described herein further calibrate the computing tool used to implement the matching in order ensure precision.

The foregoing examples have been provided merely for the purpose of explanation and are in no way to be construed as limiting of the present invention. While aspects of the present invention have been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although aspects of the present invention have been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A method for matching providers, comprising:
obtaining standardized user data from a user;
obtaining standardized provider data from providers;
generating a list of matching providers by comparing the standardized user data to the standardized provider data; and
narrowing the list of the matching providers by obtaining additional data from the user and comparing the additional data to the standardized provider data.

2. The method of claim 1, further comprising assigning values to the standardized user data in order to prioritize the standardized user data.

3. The method of claim 2, wherein the values assigned to the standardized user data are in a range of negative to positive values.

4. The method of claim 3, wherein the standardized user data includes records of data covering provider attributes.

5. The method of claim 4, wherein the assigning the values to the standardized user data includes assigning weights to the records of data.

6. The method of claim 5, wherein the records of data are assigned weights in view of importance to the user.

7. The method of claim 1, wherein the additional data is standardized and has weights assigned to the standardized additional data.

8. The method of claim 7, wherein the obtaining the additional data includes generating questions to the user to obtain the additional data.

9. The method of claim 8, wherein the narrowing the list includes eliminating the matching providers by comparing the weighted additional data to the provider data.

10. The method of claim 9, wherein the narrowing the list includes listing specific providers which remain after comparing the weighted additional data to the provider data.

11. The method of claim 1, wherein the user data is inputted by the user by providing remote access to user over a network so that the user can enter information in a collection of records in real time through a graphical user interface.

12. The method of claim 1, wherein the provider data is inputted by the providers.

13. A computer system for matching healthcare providers, comprising:
a hardware memory device that stores program instructions of a matching tool; and
a hardware processor that executes the program instructions and causes the matching tool to:
obtain user data from a user;
standardize the user data into a format;
assign values to the standardized user data in order to prioritize the standardized user data;
obtain provider data from providers;
standardize the provider data into the format;
generate a list of matching providers by comparing the prioritized user data to the provider data; and
narrow the list of the matching providers by obtaining additional data from the user and prioritizing the additional data.

14. The computer system of claim 13, wherein the user data includes records of data covering provider attributes.

15. The computer system of claim 14, wherein the assigning the values to the standardized user data includes assigning weights to the records of data.

16. The computer system of claim 13, wherein the obtaining additional data includes generating questions to the user to obtain the additional data.

17. The computer system of claim 13, further comprising assigning weights to the additional data in view of importance to the user.

18. The computer system of claim 17, wherein the narrowing the list includes eliminating the matching providers by comparing the weighted additional data to the standardized provider data.

19. A computer program product for matching healthcare providers, the computer program product comprising computer readable program instructions stored on computer readable storage medium, the computer readable program instructions being executable on a computing device to cause the computing device to:
obtain user data from a user;
weight the user data in order of importance to the user;
obtain provider data from providers;
generate a list of matching providers by comparing the weighted user data to the provider data; and
calibrate the list of the matching providers by obtaining additional data from the user and assigning weights to the additional data.

20. The computer program product of claim 19, wherein the computer readable program instructions being executable on the computing device cause the computing device to eliminate the matching providers from the list by comparing the weighted additional data to the provider data.
